(19) United States
(12) Patent Application Publication
(10) Pub. No.: US 2005/0203000 A1
(43) Pub. Date: Sep. 15, 2005

(54) Use of benzyl alcohol, and other phenolic preservatives to reduce pain during intradermal injection

(76) Inventors: Diane E. Sutter, Cary, NC (US); Ronald J. Pettis, Cary, NC (US); Scott Allen Kaestner, Durham, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/809,930

(22) Filed: Mar. 26, 2004

Related U.S. Application Data
(60) Provisional application No. 60/457,309, filed on Mar. 26, 2003.

(51) Int. Cl. A61K 38/28; A61K 31/727; A61K 31/415; A61K 31/192; A61K 31/045

(52) U.S. Cl. 514/3; 514/728; 514/690; 514/569; 514/406; 514/56

(57) ABSTRACT

Formulations and methods for reducing pain during intradermal injection, in particular the use of benzyl alcohol, phenolic agents and aromatic preservatives, antimicrobials and antioxidants for such formulations and methods.

This application claims priority to U.S. provisional application No. 60/457,309, filed Mar. 26, 2003, which is incorporated herein by reference in its entirety.

### BACKGROUND OF THE INVENTION

1. Field of the Invention

The invention relates to formulations and methods for reducing pain during intradermal injection, in particular the use of benzyl alcohol, phenolic agents and aromatic preservatives for such formulations and methods.

2. Background Information

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

In the past, intradermal delivery of drugs has not been a standard route of administration due to the limiting factors of the intradermal space (i.e. larger volumes in a limited space, difficult technique and discomfort). The few drugs commonly used for intradermal injections have been used for skin infiltration of an anesthetic agent to provide moderate duration anesthesia prior to vascular cannulation. These injections by themselves have proven to be quite painful. In many cases the amount of pain experienced is great enough to cause the recipient to forego the injection altogether. The addition of various agents to decrease pain upon injection has been limited to these anesthetic agents mainly because of the limited number of compounds delivered via the ID route. But, with the emergence of new and reliable ID delivery devices, and the improved pharmacokinetics, the desire to deliver compounds via this route has increased. Reformulating these compounds to make them “pain free” upon injection is crucial when paired with new intradermal delivery devices.

### SUMMARY OF THE INVENTION

In recent clinical trials it has been demonstrated that compounds or solutions containing preservatives in their formulation (bacteriostatic saline, insulin) have significantly lower pain scores for intradermal delivery than preservative-free solutions. The addition of benzyl alcohol (bacteriostatic saline), cresol (insulin) and aromatic preservatives to a formulation of an active compound reduces the subject's perception of pain during intradermal injection, presumably acting as a local anesthetic on the immediate tissue surface within the intradermal space, thereby making the perception of infiltration less noticeable. (It is noted that the operability of the invention is not dependent upon this being the method by which the results are achieved.) As more compounds are being investigated for future ID delivery, re-formulation and patient comfort must be considered. The addition of one of these preservatives to an active compound, which may be uncomfortable upon intradermal delivery, should decrease perception for the patient, enabling more compounds to be delivered via the intradermal route.

Pain upon microneedle/intradermal injection is associated with tissue distention or other physiological or pharmacological affect of the injectate. The addition of a desensitizer, local anesthetic or masking agent to the formulation eliminates these effects for ID administration of drugs. Previous work in this field was directed towards subsequent physical trauma such as cannula placement. The current use of the present agents is directed towards real time masking of drug and other injection effects in the ID space.

Benzyl alcohol, m-cresol and other preservatives were previously thought to be deleterious for inclusion in ID formulations based on their potential for dermal irritancy measured in animal models such as swine (Draize Irritancy Scores, NB 00-8440-01). In humans, however, these effects are not observed and beneficial results are gained in the perception of reduced pain.

Accordingly, it is an object of the invention to provide formulations and methods for intradermal injection that include a pain-reducing agent selected from the group consisting of pharmaceutically acceptable preservative agents, antimicrobial preservatives, disinfectants, antiseptics, and antioxidants. These compounds have traditionally been included in parenteral formulations for injection for the purpose of protecting, stabilizing, or otherwise preventing degradation of the drug components of an injection during storage or usage. These agents provide a chemical means of preservation by inhibiting microbial growth, and thereby, constraining decomposition of the drug or vaccine product. This preservation process is effective for control of potential pathogens, extension of product shelf life, and protecting against the deleterious consequences of microbial contamination. Antioxidants also provide chemical means of protection of the active drug process, by prevention of oxidative degradation of the active drug process. These pharmaceutical properties may either be invoked separately or in concert by a single agent. Likewise various preservative and antioxidant additives may accomplish these activities by a variety of chemical or biological mechanisms (for reference see: Dermatological Formulations, B W Barry, 1983, Marcell Dekker, New York, N.Y.). While the pharmaceutical properties of these agents are currently beneficial, their usage may not be limited by their current role. Many of the preservative, disinfecting, and antioxidant properties imparted by these agents can alternatively be accomplished by standard physical pharmaceutical processes known to those skilled in the art, such as aseptic or sterile filling and processing, terminal sterilization of products, and/or filling under inert atmosphere or vacuum, thus abrogating the need for their usage. Likewise the usage of preservatives in parenteral formulations for injection has been declining with the advent of single unit dose containers, which are accessed only once for usage, thereby minimizing or eliminating the need for an antimicrobial preservative. It is the object of this invention, to provide formulations and methods, which utilize these pharmaceutically acceptable agents for the alternative purpose of reducing the overall perception of an intradermal injection. It is the purpose of this invention to accomplish this decreased perception of ID injection using these pharmaceutically acceptable agents, where their usage is not indicated or required for achieving their preservative or antioxidant properties, or where the need for their current usage has been abrogated by other means such as effective pharmaceutical processing methods or unit-dose forms. It is to be particularly noted that modern processing methods and unit dosages have eliminated the need to include such preservatives in many pharmaceutical formulations.

The perception of ID injection (e.g. pain) may be due to physical elements of the injection process or chemical elements of the drug formulation including the active drug component or other excipients present within the drug vehicle. Physical elements of the ID injection perception process which may be masked by inclusion of an appropriate pharmaceutical additive include but are not limited to device application to the skin, insertion of needles, microneedles, microcannula or other dermal accessing means, the perception of tissue distension or increased dermal pressure upon injection, or mechanical tissue damage at the injection site caused by the injection process. Chemical and biochemical elements of the ID injection perception process which may be masked by inclusion of an appropriate pharmaceutical additive include but are not limited to: disruption of local ion channel flux, variations in local tonicity or pH at the injection site, stimulation of local or systemic immunological processes such as mast cell degranulation, stimulation of dermal nociceptors, localized vasoconstriction or vasodilatation, or localized tissue damage or necrosis due to the chemical effects.

Particularly preferred as agents are those selected from the group consisting of alcoholic preservatives (e.g. ethanol, isopropropanol, chlorbutanol, benzyl alcohol, phenoxyethanol, phenylethyl alcohol, bronopol, monothioglycerol, propylen glycol, xylitol, glycerol), acidic preservatives including inorganic and organic salts thereof (benzoic acid, lactic acid, propionic acid, sorbic acid, also sodium or potassium salts thereof), phenolic and paraben preservatives (phenol, chloroxylenol, m-cresol, o-cresol, p-cresol, chlorocresol, thymol, anisol, butylated hydroxyanisol, propyl gallate, methyl paraben, ethyl paraben, propyl paraben, butyl paraben, and halogenate derivatives thereof), quaternary ammonium preservatives (benzalkonium chloride), other preservatives (chlorhexidine, vanillin), phenolic antioxidants (butylated hydroxyanisole, butylated hydroxytoluene, nordihydroguiaretic acid, propyl gallate, pyrogallol) organic acid, alcohol, and ester antioxidants (ascorbic acid, citric acid, malic acid, sorbitol, glycerol, propylene glycol, ascorbyl palmitate) and quinone antioxidants (alpha tocopherol, hydroquinone, hydroxycoumarins).

Particularly preferred embodiments of the invention include current compounds approved for parenteral administration by the FDA or other regulatory body. It is anticipated that compounds containing benzyl, phenol, aromatic, or polyaromatic ring structures may exhibit these anesthetic and analgesic effects for ID administration. Preferred benzylic compounds (containing a benzyl structure) include benzylic alcohols and benzylic acids. Preferred phenolic compounds (containing a phenol structure) include phenolic alcohols, phenolic acids and paraben.

The concentration of such agents in the formulation is generally anticipated to be less than about 10% (w/v), preferably less than 5%, more preferably less than or equal to 2%, most preferably less than or equal to 1% (typically 0.25% or less). Useful working ranges and optimal amounts can be determined by persons of skill in the art without undue experimentation.

The above-mentioned pain-reducing agents can be included in any formulation that is suitable for, and desired to be used for intradermal injection, including diagnostic, prognostic, prophylactic and therapeutic formulations. These include diagnostic agents, drugs, and other substances which provide therapeutic or health benefits such as for example nutriceuticals. Diagnostic substances useful with the present invention include antibodies or antigens, macromolecular substances such as, for example, insulin, ACTH (e.g. corticotropin injection), luteinizing hormone-releasing hormone (e.g., Gonadorelin Hydrochloride), growth hormone-releasing hormone (e.g. Sermorelin Acetate), cholecystokinin (Sincalide), parathyroid hormone and fragments thereof (e.g. Teriparatide Acetate), thyroid releasing hormone and analogs thereof (e.g. protirelin), secretin and the like. Agents may be labeled (e.g. with radioisotopes or fluorescent tags) or unlabeled.

Therapeutic substances which can be used with the present invention include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Insulin, insulinotropin and analogs, Insulin-like growth factors, Interferons, Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Monoclonal antibodies, Peglyated antibodies, PEGylated proteins or any proteins modified with hydrophilic or hydrophobic polymers or additional functional groups, Fusion proteins, Single chain antibody fragments or the same with any combination of attached proteins, macromolecules, or additional functional groups thereof, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne Japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimer's Disease, H. Pylori, salmonella, diabetes, cancer, herpes simplex, human papilloma and the like other substances including all of the major therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, anti-depressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

Particularly preferred for use in the invention are fertility compounds (such as Antagon™, Bravelle™, Centrotide®, Follistim®, Gonal-F®, Fertinex®, Novarel™, Ovidrel® Pregnyl®, and Repronex®), antiemetics (e.g. Compazine®, Tigan®, Kytril®, Zofran®),vasoconstrictors, vasodilators, emulsions or other compositions containing particles, compositions of high viscosity, PEGylated compounds, gels, morphine and other opiod pain relievers, triptans (e.g. sumatriptan), local anesthetics (such as lidocaine), injectable sildanafil and similarly acting compounds.

It will be clear that the invention is especially useful for ID injections that have a high degree of pain, irritation or discomfort. Thus, the invention will be especially useful for ID bolus injections wherein greater than 100 ul, preferably 200 ul, more preferably 250 ul, and most preferably 500 ul is delivered per needle, and for substances that are typically irritating (e.g. heparin, low molecular weight heparin, triptan antimigrane compounds, and COX-2 inhibitors).

As used herein, “intradermal delivery” (ID) means the delivery of materials to the intradermal space and its interrogation as described by Pettis et al. in WO 02/02179 A1 (PCT/US01/20782 having a priority date of Jun. 29, 2000). “Bolus” delivery is defined as occurring over a period of less than 10 minutes.

Micro-cannula- and microneedle-based methodology and devices are described in U.S. application Ser. No. 606,909, filed Jun. 29, 2000. Standard steel cannula can also be used for intra-dermal delivery using devices and methods as described in U.S. Ser. No. 417,671, filed Oct. 14, 1999. These methods and devices include the delivery of substances through narrow gauge (30G or narrower) “micro-cannula” with a limited depth of penetration (typically ranging from 10 μm to 2 mm), as defined by the total length of the cannula or the total length of the cannula that is exposed beyond a depth-limiting hub feature.

Advantages and improvements of the present invention over currently used formulations and methods include reduced pain, enhanced delivery, and improved compliance. The additive suppresses the perception of the delivery process in a very transient and rapidly resolving manner, avoiding extended anesthesia such as that achieved with Lidocaine or other local anesthetics, which is to be avoided.

### BRIEF DESCRIPTION OF THE DRAWINGS

FIG. 1 shows the distribution of individual scores from a representative intradermal injection of preservative-free saline, as described in Example 3.

FIG. 2 shows the distribution of individual scores from a representative intradermal injection of saline containing the preservative benzyl alcohol as described in Example 4.

FIGS. 3A and 3B show calculated 95% confidence intervals for pain at intradermal device insertion (needlestick) and upon completion of saline containing the preservative benzyl alcohol as described in Example 4.

### DETAILED DESCRIPTION OF THE INVENTION METHODS

A series of prospective, randomized, open label human clinical studies were conducted. The following inclusion criteria was typically used:
18 years of age or older
In stable health status with no acute or severe illness
Willing to complete all study procedures
Available for designated study days

Persons with certain criteria (e.g. pregnancy, lactation, antibiotic therapy, etc.) were typically excluded from the study.

Typically, each participating subject received a series of ‘treatments’ consisting of intradermal infusions or injections of either sterile unpreserved isotonic saline, or sterile bacteriostatic isotonic saline for injection in the anterior thigh using microneedle-based intradermal injection devices. Participants were asked to rate the pain immediately after the device was applied to the skin, and at the end of the injection/infusion procedure. In addition, injection sites were evaluated for irritation using the Draize dermal irritation scoring scale for erythema and edema, and for the presence of any other adverse skin effects, occurring at the time of injection or developing at a later time.

Intradermal injection devices were varied in design, both for the number of microneedles inserted into the dermis and the usable length of the microneedles for a given device. Typical intradermal injection devices consisted of either 1 or 3 microcannula, with a 1-1.5 mm usable length. For example, the notation 1×1 mm indicates 1 microneedle with a 1 mm usable length. The injection sight for intradermal administration was the upper aspect of either the left or right thigh, and sites were randomized according to a predetermined dosing schedule. During intradermal administration the rate and volume of saline delivery were controlled by means of a highly accurate syringe pump, flowing at a programmed constant rate. The number of study subjects varied for different studies, as did the male/female distribution ratio. Study subjects consisted of compensated healthy male and female volunteers who were admitted based on the above representative inclusion/exclusion criteria, signed an informed consent for participation, and completed all aspects of the trial.

The examples below are from separate independent clinical studies, but are representative of outcomes routinely obtained using the device characteristics and injection methods stated. Likewise the cited examples, while not identical in method (e.g. total infusion volume) serve to illustrate the beneficial differences and altered perception outcomes encountered when an anesthetic or analgesic compound of the invention is incorporated into an intradermally administered formulation.

Pain Scores for all Studies

Pain was determined using a Gracely Box SL Scale (reference: Heft, Gracely and Dubner, Pain, 1980, 9:363-373). Pain scores were typically recorded after insertion of the intradermal delivery device, and upon complete intradermal instillation of the total saline dose. The Gracely Pain Scale is a 21 point scoring scale (0-20) with levels that go from 0 or No Pain sensation, up to 20, two levels above extremely intense.

TABLE 1
Gracely Pain Scale

| | |
|---|---|
| 20 | |
| 19 | |
| 18 | Extremely intense |
| 17 | Very intense |
| 16 | Intense |
| 15 | Strong |
| 14 | |
| 13 | Slightly intense |
| 12 | Barely strong |
| 11 | Moderate |
| 10 | |
| 9 | |
| 8 | Mild |
| 7 | Very mild |
| 6 | |
| 5 | Weak |
| 4 | Very weak |
| 3 | |
| 2 | |
| 1 | Faint |
| 0 | No pain sensation |

Typically, pain scale scores were analyzed by ANOVA using a linear-type model. Post-hoc multiple comparisons were performed if the factor effects or interaction were significant. The post-hoc comparisons helped to identify which treatments actually differ from each other and by how much on average (with 95% confidence interval). The assumptions underlying these computations were that the noise was normally distributed and that the variance was constant for all experimental conditions

### EXAMPLES 1-3

These examples were from three independently performed clinical trials using the delivery conditions stated in Table 2. Intradermal administration of various volumes of non-preserved saline was performed into the thighs of from 12-20 subjects using the indicated intradermal device and delivery conditions. The saline used in these studies did not contain any of the additives responsible for reducing the perception of the intradermal delivery process. The mean and median scores for pain perception rated on the Gracely pain scale are reported in Table 3. Scores are reported for both the insertion of the intradermal delivery microcannula alone prior to administration of any saline, and for end of injection pain perception, which rates the overall pain of the intradermal saline delivery process.

These examples illustrate that the insertion of a microneedle based delivery device typically ranks very low on the pain perception scale. Most mean and median device insertion scores ranked between 0-2 on the Gracely perception scale, near the “faint” reporting region. Upon complete administration of the saline dosage all studies showed a statistically significant increase in reported perception scores. Mean scores ranked between 4-9 for the intradermal administration process, corresponding to the “weak” to “mild” range of the Gracely scale. These independently gathered data indicate that although the device systems may be minimally perceptible, the administration process is responsible for the increase of perceived pain, even for low administration volumes.

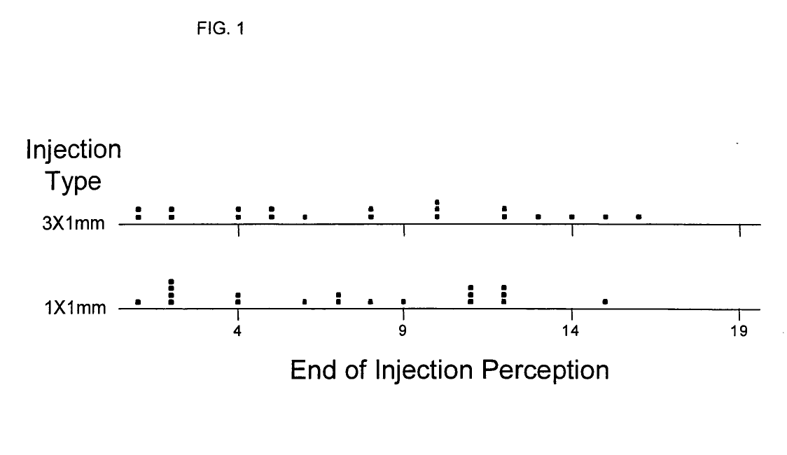

In addition a dotplot showing the distribution of end of injection scores from all subjects in Example 3, Conditions E and F is shown in FIG. 1. Statistical ANOVA analysis of the response data, indicate a statistically significant subject-to-subject difference for end of injection pain perception. This is reflected in the wide distribution of perception scores seen in FIG. 1. This subject-based difference was also statistically significant for the conditions tested in Examples 1 and 2, with a similar wide distribution for end-of-injection scoring (data not shown).

TABLE 2

| | | | Saline | Saline | |
|---|---|---|---|---|---|
| | | Saline | Delivery | Delivery | |
| | Device | Volume | Rate | Duration | # Study |
| | Configuration | (μL) | (μL/min) | (min) | Subjects |
| Example 1 | | | | | |
| A | 1 × 1 mm | 250 | 50 | 5 | 12 |
| B | 1 × 1 mm | 300 | 50 | 5.5 | 12 |
| Example 2 | | | | | |
| C | 3 × 1 mm | 100 | 50 | 2 | 16 |
| D | 3 × 1 mm | 100 | 100 | 1 | 16 |
| Example 3 | | | | | |
| E | 1 × 1 mm | 100 | 100 | 1 | 20 |
| F | 3 × 1 mm | 100 | 100 | 1 | 20 |

TABLE 3

| | |
|---|---|
| Intradermal Device | End of |
| Insertion Pain | Injection Pain |

| Condition | Mean | Median | Mean | Median |
|---|---|---|---|---|
| Example 1 | | | | |
| A | 1.0 | 0 | 6.7 | 8 |
| B | 1.4 | 0 | 4.8 | 5 |
| Example 2 | | | | |
| C | 3.7 | 3 | 7.3 | 7.5 |
| D | 1.9 | 0 | 8.4 | 10 |
| Example 3 | | | | |
| E | 1.2 | 1 | 7.3 | 7 |
| F | 2.2 | 1 | 7.9 | 8 |

### EXAMPLE 4

In an independently performed study in 20 subjects, intradermal injections were made using sterile saline for injection containing 0.9% benzyl alcohol, under the conditions of Table 4. The study was performed using methods and techniques as reported above and similar to those used in Examples 1-3, with the principle variation being the use of a pain-reducing additive in the administered solution.

TABLE 4

| | | | Saline | Saline | |
|---|---|---|---|---|---|
| | | Saline | Delivery | Delivery | |
| | Device | Volume | Rate | Duration | # Study |
| | Configuration | (μL) | (μL/min) | (min) | Subjects |
| Example 4 | | | | | |
| G | 3 × 1.0 mm | 600 | 100 | 6 | 20 |
| H | 3 × 1.5 mm | 600 | 100 | 6 | 20 |
| I | 3 × 1.0 mm | 400 | 100 | 4 | 20 |
| J | 3 × 1.5 mm | 400 | 100 | 4 | 20 |

TABLE 5

| | |
|---|---|
| Intradermal Device | End of |
| Insertion Pain | Injection Pain |

| Condition | Mean | Median | Mean | Median |
|---|---|---|---|---|
| Example 4 | | | | |
| G | 1.8 | 1.0 | 1.4 | 0 |
| H | 2.2 | 1 | 0.8 | 0 |
| I | 1.8 | 1 | 0.7 | 0 |
| J | 1.4 | 0 | 1 | 0 |

It can be seen that the pain scores after infusion process for 1 mm and 1.5 mm needle length intradermal delivery devices were much lower in this Example compared to the prior three examples, despite an increase in administered volume. In fact mean pain scores after administration ranked near the “faint” range of the Gracely perception scale, while median scores ranked as “no pain perception”.

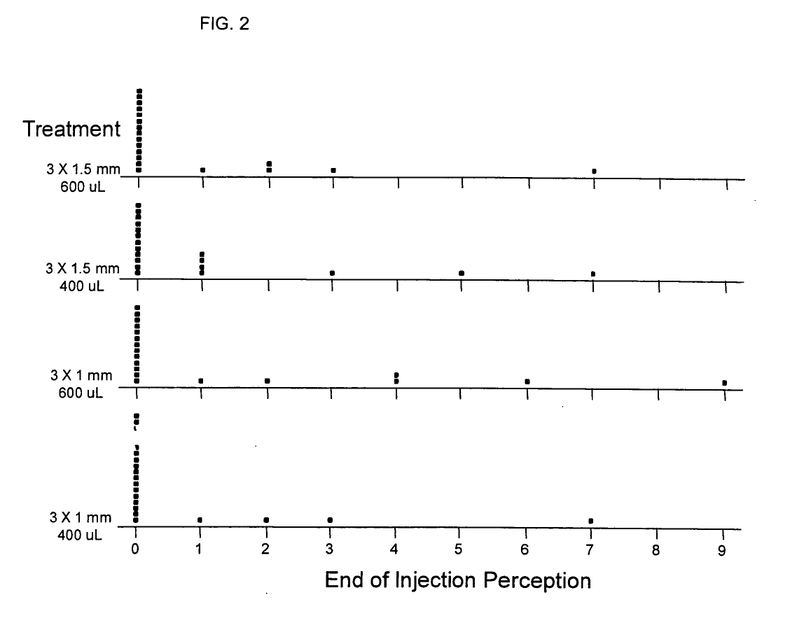

A dotplot showing the distribution of individual pain scores at end of injection for conditions G-J of Example 4 is shown in FIG. 2. Statistical ANOVA analysis of the perception score was performed on the data from Example 4. The ANOVA model included subject-to-subject differences, nurse effect, time recorded (needle stick or after entire dose), order of injection, leg (R or L), needle lengths, volume and the needle lengths by volume interaction. The ANOVA results showed that the average pain after the needle stick was significantly higher by 0.8 pain scale units (with 95% CI of (0.3, 1.4)) than the average pain after the entire dose. The only other detected significant effect was the subject effect at either needle placement or end-of-dose. Overall, the subject variability was also decreased as shown by the tight clustering of reported scores seen in FIG. 2.

Delivery of high dose volume to the ID space in the human clinical trials of Example 4 resulted in an exceptionally low perception of pain when including pain reducing agents of the invention. Scores were substantially lower than all previous delivery to this tissue space and even below that of microneedle insertion. All scores ranked as imperceptible or nearly imperceptible on a 21-point Gracely Pain Scale.

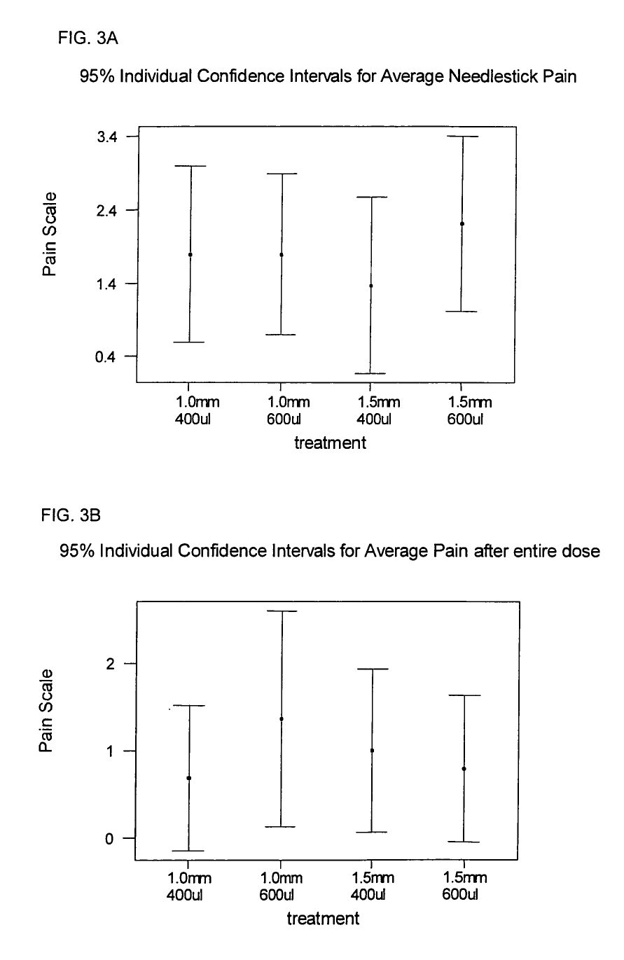

A comparison of average pain per injection type is shown in FIGS. 3A and 3B FIGS. 3A and 3B show individual 95% confidence intervals for needle stick pain and pain after the administration of the entire dose per device. It should be noted that in the previous examples 1-3, participant's rated the pain after the entire dose had been delivered as significantly higher than the pain after the initial needle stick.

Each of the aforementioned examples employed injectates that were “pharmaceutically accepted vehicles without biologically active medicaments”, e.g., isotonic saline. The injectate used in Example 4 was Abbott Laboratories Bacteriostatic 0.9% sodium chloride, injection, USP. The injectate of Example 4 contained 9 mg/ml of benzyl alcohol in addition to saline. Examples 1 through 3 used an injectate which was a preservative-free saline that contains no benzyl alcohol.

The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described. Patents and references cited herein are hereby incorporated by reference.

1. A composition for intradermal injection that includes a pain-reducing agent selected from the group consisting of pharmaceutically acceptable preservatives, antimicrobials, disinfectants, and antioxidants.

2. The composition of claim 1 wherein the agent is selected from the group consisting of benzylic compounds.

3. The composition of claim 2 wherein the agent is a benzylic alcohol or benzylic acid.

4. The composition of claim 3 wherein the agent is benzyl alcohol.

5. The composition of claim 1 wherein the agent is a phenolic agent.

6. The composition of claim 5 wherein the agent is a phenolic alcohol, phenolic acid or paraben.

7. The composition of claim 1 wherein the agent is cresol.

8. The composition of claim 1 wherein the agent comprises an aromatic ring structure.

9. The composition of claim 1 wherein the agent is an organic alcohol.

10. The composition of claim 1 wherein the agent is a quinone.

11. The composition of claim 2 wherein the agent is present in a concentration of<1%

12. The composition of claim 1 that comprises a pharmaceutical agent selected from the group consisting of insulin, heparin, low molecular weight heparin, triptan antimigraine compounds, and COX-2 inhibitors.

13. A method of reducing pain during intradermal injection of a composition, comprising including in said composition a pain-reducing agent selected from the group consisting of pharmaceutically acceptable preservatives, antimicrobials, disinfectants, and antioxidants.

14. The method of claim 13 wherein the agent is selected from the group consisting of benzylic compounds.

15. The method of claim 14 wherein the agent is a benzylic alcohol or benzylic acid.

16. The method of claim 15 wherein the agent is benzyl alcohol.

17. The method of claim 13 wherein the agent is a phenolic agent.

18. The method of claim 17 wherein the agent is a phenolic alcohol, phenolic acid or paraben.

19. The method of claim 13 wherein the agent is cresol.

20. The method of claim 13 wherein the agent comprises an aromatic ring structure.

21. The method of claim 13 wherein the agent is an organic alcohol.

22. The method of claim 13 wherein the agent is a quinone.

23. The method of claim 13 wherein the agent is present in a concentration of<1%.

24. The method of claim 13 that wherein the agent is selected from the group consisting of insulin, heparin, low molecular weight heparin, triptan antimigraine compounds, and COX-2 inhibitors.

25. The method of claim 13 wherein the intradermal injection is a bolus injection.

26. The method of claim 25 wherein the bolus injection is administered in a volume of greater than 100 ul per needle.

27. The method of claim 26 wherein the bolus injection is administered in a volume equal to or greater than 100 ul per needle.

28. The method of claim 27 wherein the bolus injection is administered in a volume equal to or greater than 200 ul per needle.

29. The method of claim 28 wherein the bolus injection is administered in a volume equal to or greater than 250 ul per needle.

30. The method of claim 29 wherein the bolus injection is administered in a volume equal to or greater than 500 ul per needle.

31. In a method comprising the intradermal injection of a composition into the skin of a mammal, the improvement comprising the inclusion in said composition of a pain reducing agent selected from the group consisting of pharmaceutically acceptable preservatives.

32. The method of claim 31 wherein the agent is selected from the group consisting of benzylic compounds.

33. The method of claim 32 wherein the agent is a benzylic alcohol or benzylic acid.

34. The method of claim 33 wherein the agent is benzyl alcohol.

35. The method of claim 31 wherein the agent is a phenolic agent.

36. The method of claim 35 wherein the agent is a phenolic alcohol, phenolic acid or paraben.

37. The method of claim 31 wherein the agent is cresol.

38. The method of claim 31 wherein the agent comprises an aromatic ring structure.

39. The method of claim 31 wherein the agent is an organic alcohol.

40. The method of claim 31 wherein the agent is a quinone.

41. The method of claim 31 wherein the agent is present in a concentration of<1%.

42. The method of claim 31 that wherein the agent is selected from the group consisting of insulin, heparin, low molecular weight heparin, triptan antimigraine compounds, and COX-2 inhibitors.

43. The method of claim 31 wherein the intradermal injection is a bolus injection.

44. The method of claim 43 wherein the bolus injection is administered in a volume of greater than 100 ul per needle.

45. The method of claim 44 wherein the bolus injection is administered in a volume equal to or greater than 100 ul per needle.

46. The method of claim 45 wherein the bolus injection is administered in a volume equal to or greater than 200 ul per needle.

47. The method of claim 46 wherein the bolus injection is administered in a volume equal to or greater than 250 ul per needle.

48. The method of claim 47 wherein the bolus injection is administered in a volume equal to or greater than 500 ul per needle.

49. The method of claim 13 wherein the composition is administered for a therapeutic, diagnostic or prognostic purpose.

50. The method of claim 31 wherein the composition is administered for a therapeutic, diagnostic or prognostic purpose.
